(19) United States
(12) Patent Application Publication
(10) Pub. No.: US 2006/0179554 A1
(43) Pub. Date: Aug. 17, 2006

(54) Goggle with removable foam lining

(75) Inventor: David A. Barton, Cardiff, CA (US)

Correspondence Address: Kit M. Stetina, Esq.;STETINA BRUNDA GARRED & BRUCKER, Suite 250, 75 Enterprise, Aliso Viejo, CA, 92656, US

(73) Assignee: Spy Optic, Inc.

(21) Appl. No.: 11/057,461

(22) Filed: Feb. 14, 2005

(51) Int. Cl. A61F 9/02

(52) U.S. Cl. 2/426

(57) ABSTRACT

Provided is a goggle comprising a face shield attached to a first subframe and a foam assembly having a foam element and second subframe. The foam assembly is removably attachable to the first subframe such that variously configured foam assemblies are interchangeable with the first subframe and face shield. More particularly, the foam element may define a proximal surface that is sized and configured to fit a wide face, a narrow face, a short face, a long face and/or various combinations thereof. Also, the foam assembly may be sized and configured to be compatible with different helmets. A plurality of these variously configured foam assemblies may be provided with a single first subframe and face shield such that a wearer may select a desired face shield and an appropriate foam assembly in order to accommodate the wearer's uniquely shaped face and/or helmet.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

The present invention relates to goggles and, more particularly, to goggles having a first subframe which is removably attachable to and interchangeable with a plurality of variously configured foam assemblies.

A goggle is necessary for and should be worn during certain physical activities such as snowboarding, skiing, hiking, motocross racing, mountain biking and/or other types of physical activities where environmental debris may be projected toward the participant's eyes. The goggle is typically worn during the entire duration of the physical activity which may extend up to eight or more hours. Accordingly, it should be comfortable to wear. To provide for a comfortable fit between the goggle and the wearer's face, the goggle typically includes a soft foam lining which provides a comfortable interface between the goggle and the wearer's face. Unfortunately, the foam lining may wear down over time and the goggle may be uncomfortable to wear. Moreover, certain physical activities such as motocross racing also require the participant to wear a helmet over which the goggle is worn. As such, the goggle must be both comfortable to wear and compatible with the many different helmets available on the market.

To accommodate children and adults, goggles are supplied at retail establishments in various sizes such as youth and adult. Moreover, within the adult category, they may be provided in three or more sizes such as small, medium, or large. The variety of sizes in which goggles are provided allows the wearer to select the goggle which best fits their face structure. For example, a wearer having a relatively wide face may select a large-sized goggle. In contrast, a wearer having a relatively narrow face may select a smaller-sized goggle. However, if the wearer has a relatively wide face but a small cheekbone structure, the wearer may have to settle for the large-sized goggle because a smaller goggle size may not fit the wearer's face. Likewise, if the wearer has a relatively narrow face with high cheekbones, a more comfortable fit may be provided using a medium or large-sized goggle. However, in order to obtain a relatively tight fit between the goggle and the wearer's face, the wearer may have to settle for a smaller-sized goggle.

Accordingly, there is a need in the art to increase the comfort level of a goggle by providing a closer fit between the goggle and the wearer's face. Furthermore, there exists a need in the art for a goggle that is of simple construction and of relatively low cost. There also exists a need in the art for a goggle that may fit many different face structures and many different helmets. Also, there is a need in the art to address worn down foam linings such that there may be a comfortable interface between the goggle and the wearer's face.

### BRIEF SUMMARY OF THE INVENTION

In accordance with an aspect of the present invention, there is provided a goggle comprising a face shield and a first subframe which are each attachable to one other. The goggle may further comprise a foam assembly (or seal assembly) having a second subframe and a foam element. The second subframe of the foam assembly may be removably attachable to the first subframe. In this regard, the first subframe and face shield are removably attachable to (i.e., interchangeable with) a plurality of variously configured foam assemblies. The various configurations of the foam assembly are customized to fit variously shaped faces and are intended to absorb sweat, keep out debris and allow the goggles to float within a helmet when worn in conjunction therewith. By way of example and not limitation, the foam assemblies may be fabricated to fit a wide face, a narrow face, a short face, a long face, a face with high cheek bones, a face with small cheek bones and various combinations thereof. Furthermore, the various configurations of the foam assembly may be customized to be compatible with different helmets, to be used in different environmental conditions and/or to allow foam accessories to be attached thereto.

The removable attachable feature between the first subframe and the foam assembly may occur at the interface between the first subframe and the second subframe of the foam assembly. In particular, the first subframe may have a flange about a periphery thereof wherein the flange may have an L-shaped cross sectional configuration. Correspondingly, the second subframe of the foam assembly may have a lip formed about a periphery of the second subframe wherein the lip may have a mating L-shaped cross sectional configuration. The flange and lip may be sized and configured to engage one another such that the lip is stretchable around the flange. Accordingly, the foam assembly may be removed and attached to the first subframe as desired by disengaging or engaging the lip and flange. In this regard, a plurality of foam assemblies, each sized and configured to fit the particular structure of a wearer's face, helmet, and/or environmental condition, may be interchangeable with the first subframe and face shield.

### BRIEF DESCRIPTION OF THE DRAWINGS

An illustrated and presently preferred embodiment of the invention is shown in the accompanying drawings in which:

FIG. 1 is a perspective view of a goggle having a foam assembly, a first subframe and a face shield wherein the foam assembly may be removably attachable to the first subframe;

FIG. 2 is an exploded view of FIG. 1 and illustrating the foam assembly being removed from the first subframe;

FIG. 3 is an exploded rear perspective view of FIG. 1 and further illustrating a flange extending about a periphery of one of the subframes attached to the face shield;

FIG. 4 is an exploded top view of FIG. 1;

FIG. 5 is a front view of the foam assembly further illustrating a lip extending about the second subframe periphery;

FIG. 6 is a side view of FIG. 5;

FIG. 7 is a cross sectional side view of FIG. 4 and illustrating engagement between the flange lip and the second subframe periphery mating lip;

FIG. 8 is an exploded view of the goggle illustrating posts disposed about a periphery of the second subframe; and

FIG. 9 is an exploded rear perspective view of the goggle of FIG. 8 illustrating apertures sized and configured to receive the posts.

### DETAILED DESCRIPTION OF THE INVENTION

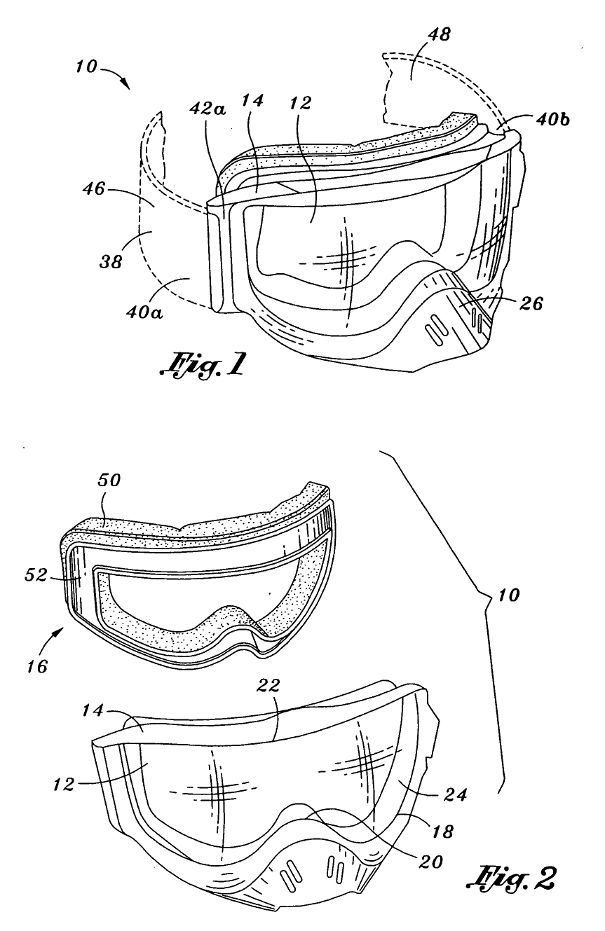

Referring now to the drawings wherein the drawings are for the purposes of illustrating the preferred embodiments of the present invention only, and not for the purposes of limiting the same, FIG. 1 is a perspective view of a goggle 10 configured for protecting a person's eye from debris which may be projected toward the person's eye. In this regard, it is contemplated within the scope of the present invention that the goggle 10 may be fabricated, designed or otherwise fitted to fit other mammals. For example, the goggle 10 may be designed and fabricated to fit around a dog's head in order to protect the dog's eyes from debris which may be projected theretoward.

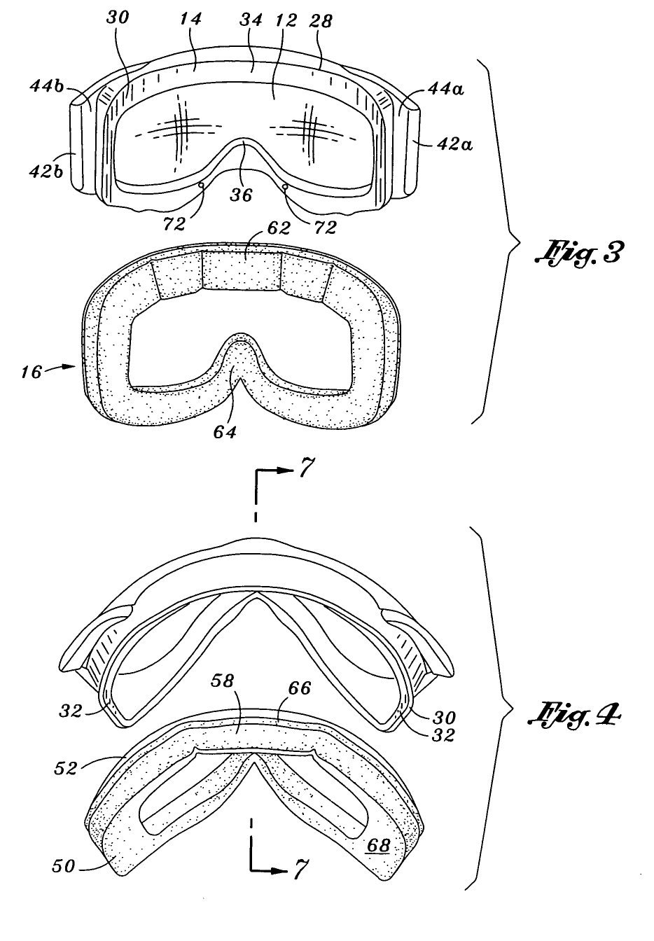

In an aspect of the present invention, the goggle 10 may comprise a face shield 12, a first subframe 14 and a foam assembly 16, as shown in FIGS. 2 and 3. The face shield 12 may be fabricated from a transparent, semi-transparent or one way transparent material for allowing a goggle wearer to maintain visual contact with the wearer's environment. By way of example and not limitation, the wearer may be a snowboarder or motocross racer traversing a mountainside. In this scenario, the snowboarder or motocross racer (i.e., the wearer) may wear the goggle 10 in order to prevent debris such as rocks and snow from entering into the snowboarder's or racer's eyes but allowing the snowboarder to maintain awareness of or visual contact with the wearer's surrounding (i.e., moguls, trees and other people).

The face shield 12 may be configured to extend across the wearer's eyes in a curved fashion when worn. The face shield 12 may also be configured to extend across the wearer's eyes in an amount equal to or greater than an effective field of vision of the wearer in both the lateral and horizontal directions with respect to the wearer's face. The face shield 12 may additionally define a periphery 18 (see FIG. 2) wherein a bottom edge 20 of the periphery 18 is sized and configured to fit around the wearer's nose. A top edge 22 of the face shield 12 may additionally be sized and configured to fit horizontally across a wearer's forehead. The face shield 12 may be fabricated from a plastic material via an injection molding process as well as from other materials and processes.

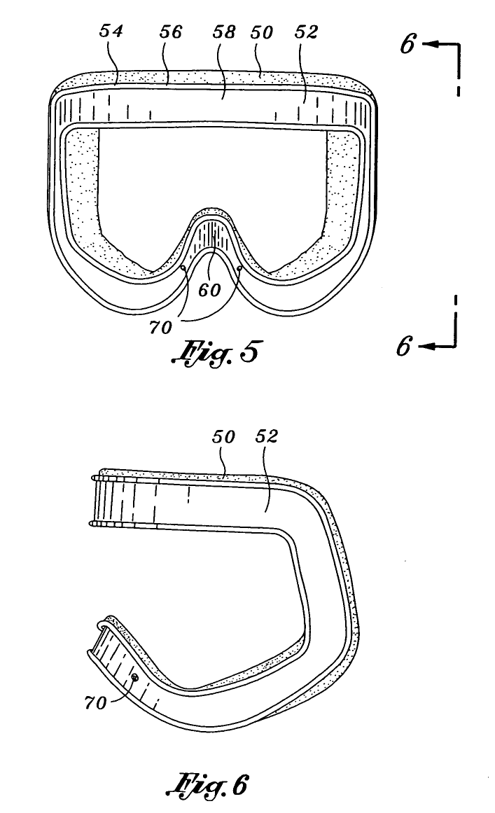

The first subframe 14 may be attached to the face shield 12, as shown in FIGS. 1-3. In particular, the face shield 12 may be attached to the first subframe 14 via a formed groove 23 (see FIG. 7) extending about an inner surface 24 of the first subframe 14. The groove 23 may be sized and configured to receive the face shield periphery 18. Optionally, the first subframe 14 may further have formed therein a nose guard and/or mouth guard 26 to prevent upwardly projected debris from entering the wearer's nose and/or mouth during engagement of physical activity (e.g., snowboarding or motocross racing). The first subframe 14 may further define a first subframe periphery 28. Formed about the first subframe periphery 28 may be a flange 30, as shown in FIGS. 3 and 4. The flange may extend continuously about the first subframe periphery 28. Although not shown, it is contemplated that the flange 30 may be formed about the first subframe periphery 28 at intermittent intervals therealong. The flange 30 may have a T-shaped cross sectional configuration. In the alternative the flange 30 may have an L-shaped cross sectional configuration. Although the L-shaped flange 30 is shown in FIGS. 4 and 7 as having a lip 32 (see FIG. 4) extending outwardly about the first subframe periphery 28, the lip 32 may extend inwardly about the first subframe periphery 28.

Similar to the configuration of the top edge 22 and bottom edge 20 of the face shield periphery 18, the first subframe periphery 28 may also have a top edge 34 and a bottom edge 36 which are sized and configured to fit across the wearer's forehead and nose, respectively. As shown in FIG. 3, the bottom edges 20, 36 of the face shield 12 and first subframe 14 may have a corresponding inverted “V” configuration. The first subframe 14 may be fabricated from a plastic material via an injection molding process. The first subframe material may be transparent, semi-transparent or one way transparent material.

A strap 38, as shown in FIG. 1, may be attached to the face shield 12, first subframe 14 or foam assembly 16, although it is preferred that the strap 38 is attached to the first subframe 14 such that the first subframe 14 may apply pressure to the foam assembly 16 and onto the wearer's face when the goggle 10 is worn by the wearer. The strap 38 may be fabricated from a soft, quick drying, and/or elastic material such as neoprene, spandex, cotton, etc. The strap 38 may be formed from a unitary material with its distal ends 40a, b being looped about posts 42a, b (see FIG. 3) formed along lateral edges 44a, b (see FIG. 3) of the first subframe 14. In the alternative, the strap 38 may comprise a first member and a second member 46, 48 (see FIG. 1) wherein the first member 46 may be attached to the first subframe 14 and the second member 48 may also be attached to the first subframe 14 at opposed lateral edges 44a,b of the first subframe 14. The first and second members 46, 48 may define distal ends and may be removably attachable to one another via hooks and loops and/or an adjustable locking mechanism disposed at the distal ends of the first and second members 46, 48.

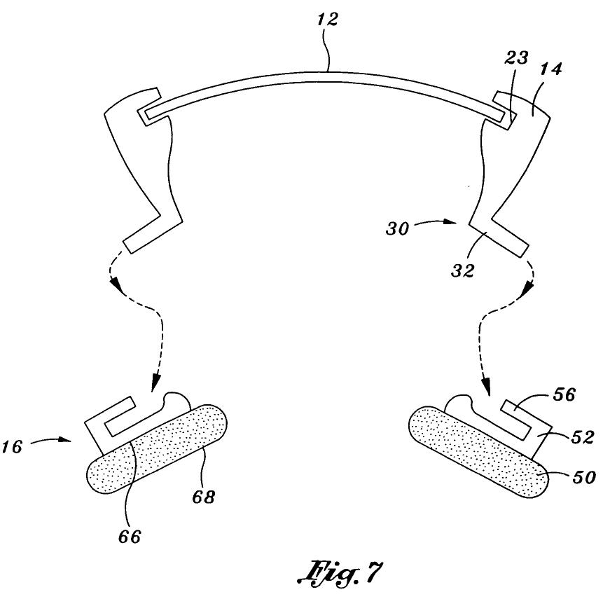

The foam assembly 16 may comprise a foam element 50 and second subframe 52, as shown in FIGS. 2 and 4-7. Although the various aspects of the present invention are discussed in relation to the foam element 50, it is also contemplated that the foam element 50 may be replaced with an elastomeric material (e.g., face seal of diver's mask) or other type of material which can form a seal with the wearer's face. In this case, the foam assembly 16 may be referreed to as a seal assembly 16. Furthermore, it is also contemplated within the scope of the present invention that the aspects of the second subframe 52 discussed below may be incorporated into the foam element 50 or replacement material to eliminate the second subframe altogether. The foam element 50 may be attached to the second subframe 52 via an adhesive, hot melt or other permanent or temporary attachment mechanism disposed therebetween. The second subframe 52 may define a periphery 54 having a mating lip 56 (see FIGS. 5 and 7) formed about the second subframe periphery 54. The mating lip 56 extending about the second subframe periphery 54 may be sized and configured to engage the flange lip 32 (see FIG. 7) of the first subframe 14, as shown in FIG. 7. The cross sectional configuration of the second subframe 52 may correspond to and may be configured complementary to the selected cross sectional configuration of the first subframe 14. For example, if the first subframe cross sectional configuration is L-shaped, then the cross sectional configuration of the second subframe 52 may be a mating L-shape, as shown in FIG. 7. The second subframe 52 may be fabricated from a pliable resilient material such as rubber such that the mating lip 56 may readily engage or latch onto the flange lip 32.

The second subframe 52 may have a top edge 58 and a bottom edge 60 which may be sized and configured to fit across the wearer's forehead and nose. Furthermore, the bottom edge 60 of the second subframe 52 may have an inverted V-shaped configuration (see FIG. 5). Moreover, as shown in FIG. 4, the top edge 58 of the second subframe 52 may have a curved configuration.

The foam element 50, which may be attached to the second subframe, may be fabricated from a material that is generally comfortable to wear, resilient and elastic. The foam may be fabricated from an open cell or a closed cell foam. The foam element 50 may be configured to form a seal between the wearer's face and the second subframe when the goggle is worn by the wearer. The foam element 50 may have a silhouette that is comparable to that of the second subframe 52. Moreover, the foam element 50 may have a top edge 62 and lower edge 64 which are sized and configured to fit around the wearer's forehead and nose. The foam element 50 may further define a distal surface 66 and a proximal surface 68 (see FIGS. 4 and 7) wherein the distal surface 66 is attachable to the second subframe 52.

The proximal surface 68 may be sized and configured to fit the wearer's face. In this regard, the foam proximal surface 68 may be customized to match a particular type of head configuration and, more accurately, to a particular type face configuration or to unique features of the wearer's face. For example, the proximal surface 68 may be sized and configured to fit a wide face, a narrow face, a long face, a short face, a face with high cheek bones, a face with a large jaw or a face with a small jaw and various combinations thereof. Nonetheless, despite the configuration of the foam proximal surface 68, the foam distal surface 66 may retain the same shape to match that of the second subframe 52. Accordingly, a first unique advantage of the present invention over the prior art is that a single combination of the face shield 12, first subframe 14 and strap 38 may be fit to a variety of head structures via interchanging the foam assembly 16 (i.e., second subframe and foam element 50) so as to fit the unique head configuration of the wearer. Advantageously, this results in a highly customized and comfortable fit to the wearer. Furthermore, as discussed above, this aspect provides additional comfort above and beyond the comfort provided by the foam material in relation to its type and/or density.

A second unique advantage of the interchangeability of the foam assembly 16 with the first subframe 14 and face shield 12 is that, instead of stocking many different goggle sizes, a retail establishment may only need to stock a single sized first subframe 14 and face shield 12 and a plurality of different foam assemblies thereby reducing its inventory. A third unique advantage of the interchangeability of the foam assembly 16 with the first subframe 14 is that, as the foam element 50 degrades, the wearer may purchase a replacement foam assembly 16—the foam assembly 16 is disposable—so as to restore the original comfort and fit of the goggle 10. Alternatively, the foam element 50 of the foam assembly 16 may be replaced—the foam element 50 is disposable—so as restore the original comfort and fit of the goggle 10.

A fourth unique advantage of the interchangeability of the foam assembly 16 with the first subframe 14 is that a plurality of foam assemblies 16 may be sized and configured to be compatible with a plurality of helmets such that the foam assembly fits snugly into a face opening of the helmet. Also, the plurality of foam assemblies sized and configured to be compatible with the plurality of helmets may also be sized and configured to fit a particular face type such that the first subframe 14 and face shield 12 are compatible with both different face types and different helmets.

The attachment between the first subframe 14 and the second subframe 52 may be accomplished via a plurality of posts 70 (see FIGS. 5 and 6 and FIG. 8) formed about the second subframe periphery 54 mating with a plurality of post receivers 72 (see FIG. 3 and FIG. 9) formed about the first subframe periphery 28. The plurality of posts 70 and post receivers 72 are sized, configured and positioned about the respective ones of the peripheries 54, 28 so as to be aligned when the first and second subframes 14, 52 engage one another.

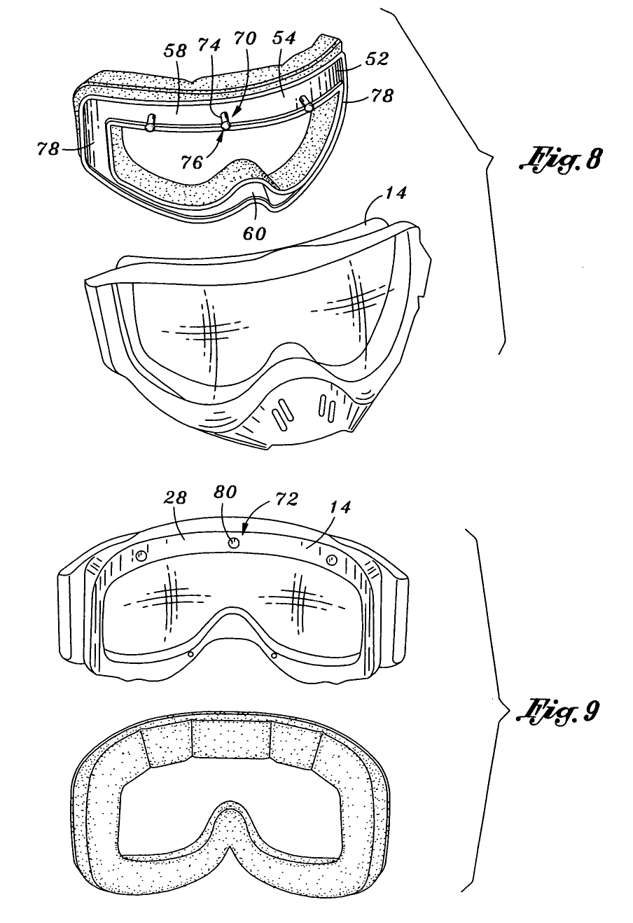

As shown in FIGS. 8 and 9, the post 70 may have a cylindrical base 74 with a flared head 76. The post 70 may be fabricated from a resilient material which may allow the posts 70 and post receivers 72 to engage and disengage multiple times. FIG. 8 illustrates three posts 70 formed along the top edge 58 of the second subframe 52. However, it is also contemplated that the bottom edge 60 and lateral edges 78 may have posts 70 formed thereon. The post receivers 72 may be configured as apertures 80 formed within the first subframe 14, as shown in FIG. 9.

A recessed portion of the post receiver 72 may be formed within the first subframe 14 and may be sized and configured to receive the post flared head 76. In other words, the recessed portion may be expanded to a diameter equal to the flared head 76 whereas the aperture 80 may have a diameter equal to a diameter of the cylindrical base 74. This provides interlocking engagement between the posts 70 and post receivers 72. Alternatively, as shown in FIGS. 3, 5 and 6, the posts 70 may have a cylindrical configuration sized and configured to fit with the post receivers 72.

This description of the various embodiments of the present invention is presented to illustrate the preferred embodiments of the present invention, and other inventive concepts may be otherwise variously embodied and employed. The appended claims are intended to be construed to include such variations except insofar as limited by the prior art.

What is claimed is:

1. A goggle worn on a person's face having unique features to protect the person's eyes from debris, the goggle comprising:
a. a face shield for blocking debris projected toward the person's eyes;
b. a first subframe attached to the face shield and defining a periphery; and
c. a seal assembly defining a periphery and a proximal surface, the proximal surface sized and configured to the unique features of the person's eyes and the seal assembly periphery being removeably attachable to the first subframe periphery.

2. The goggle of claim 1 wherein the first subframe has a flange about the first subframe periphery and the seal assembly has a mating lip about the seal assembly periphery, and the flange is removeably attachable to the lip.

3. The goggle of claim 1 wherein the seal assembly has a plurality of posts about the seal assembly periphery and the first subframe has a plurality of post receivers, and the posts are removably attachable to respective post receivers.

4. The goggle of claim 1 wherein the seal assembly is a foam assembly.

5. The goggle of claim 1 wherein the seal assembly is fabricated from a unitary material.

6. The goggle of claim 1 wherein the seal assembly comprises a second subframe defining a periphery and an elastomeric material, and the second subframe periphery is removeably attachable to the first subframe periphery.

7. The goggle of claim 6 wherein the elastomeric material is selected from the group consisting of foam and rubber.

8. A goggle system for protecting a person's eyes from debris, the goggle comprising:
a. a face shield for blocking debris projected toward the person's eyes;
b. a first subframe attached to the face shield and defining a periphery; and
c. a plurality of seal assemblies, each seal assembly defining a periphery, each seal assembly periphery being removeably attachable to the first subframe periphery.

9. The goggle of claim 8 wherein the seal assemblies define proximal surfaces which are sized and configured to fit a plurality of different face types.

10. The goggle of claim 9 wherein the face types are a wide face, a narrow face, a long face and a short face.

11. The goggle of claim 8 wherein the seal assemblies have different densities.

12. The goggle of claim 8 wherein the seal assemblies have different thicknesses.

13. The goggle of claim 8 wherein each seal assembly comprises a foam element and a second subframe, and the second subframe is removably attachable to the first subframe.

14. The goggle of claim 8 wherein the seal assembly is fabricated from a unitary material.

15. The goggle of claim 8 wherein the seal assembly is a foam assembly.

16. The goggle of claim 8 wherein the seal assemblies are sized and configured to be compatible with a plurality of helmets.
